(19) United States
(12) Patent Application Publication
(10) Pub. No.: US 2019/0314544 A1
(43) Pub. Date: Oct. 17, 2019

(54) MANUFACTURING PROCESS FOR AN ADHERENT SILICONE HEALING TAPE/PAD/DRESSING OF SPECIFIC SHAPES

(71) Applicants: Maria Cristina De Paula Mesquita, Uberlândia (BR); Vania De Fatima Diniz Ferreira, Uberlândia (BR)

(72) Inventors: Luiz Ricardo Goulart Filho, Uberlândia (BR); Roberta Rezende Rosa, Uberlândia (BR)

(21) Appl. No.: 16/217,999

(22) Filed: Dec. 12, 2018

(30) Foreign Application Priority Data
Apr. 11, 2018 (BR) 10 2018 007306 0

(51) Int. Cl. A61L 15/58; A61L 15/44; A61F 13/02

(52) U.S. Cl. CPC A61L 15/585; A61L 15/44; A61F 13/0253; A61F 13/0289; A61F 2013/00157; A61L 2300/428; A61L 2300/426; A61L 2300/41; A61L 2300/412; A61L 2300/442

(57) ABSTRACT

A new MANUFACTURING PROCESS FOR AN ADHERENT SILICONE HEALING TAPE/PAD/DRESSING OF SPECIFIC SHAPES, for application in the medical area aiming to provide greater results in the healing of keloid scars as a fibroproliferative disorder, since the following specific properties make silicone useful for dressings: safe, low thermal conductivity, low chemical reactivity, low toxicity and rare adverse reactions, as the silicone cannot be absorbed into the body, repels the water and creates a watertight seal, it does not support microbial growth, has high gas permeability, and is adherent to dry skin.

### CROSS REFERENCE TO RELATED APPLICATION

This applications takes the benefit of and claims priority to Brazilian Patent Application Serial No. 10 2018 007306 0 filed on Apr. 11, 2018, the contents of which are herein incorporated by reference.

### BACKGROUND OF THE INVENTION

### Field of the Invention

The present invention focus on a new MANUFACTURING PROCESS FOR AN ADHERENT SILICONE HEALING TAPE/PAD/DRESSING OF SPECIFIC SHAPES, with application in the medical area aiming to provide a greater preventive, aesthetic and functional result of the cicatrization process of widened, hypertrophic and keloidian scars that trigger fibroproliferative disorders, since the following specific properties make silicone useful for dressings: safe, presenting low thermal conductivity, low chemical reactivity and low toxicity, with rare adverse reactions, as the silicone cannot be absorbed into the body, repelling the water and maintaining the moisture in the skin, and also, it does not promote the microbial growth.

### Description of the Related Art

Different types of dressing processes and silicone tapes are known today, which may be mentioned: a—Primary wound contact layers, which are composed of a non-absorbent polyamide web, allowing the exudate to pass to an absorbent dressing, useful for superficial wounds such as: skin tears, skin abrasions, surgical wounds, second-degree thermal burns, lacerations, leg ulcers, pressing wounds, negative pressure wound therapy; b—Foam dressings with silicone adhesive: thin adhesive layer of silicone that absorbs fluid vertically, absorbent clothing, minimizes exudate contact with the skin around the wound, recommended for low-high exuding wounds, leg ulcers, pressing wounds, cuts and abrasions, surgical wounds, small thermal burns, friable or delicate skin; c—Silicone gel sheets: thick sheet of silicone gel that does not require a secondary dressing, designed for use on healed wounds to minimize hypertrophic/keloid scars. The properties of the primary dressing silicone sheets include: minimal pain and trauma on dressing changes, no extra fixation is required, thin, flexible and discreet, can be used in daily activities, can be reapplied, can be cut to size, and waterproofed.

It is well known that for the performance of current dressings or tapes the utmost care is required, since the healing process for each case depends on the correct application of the type of wound dressing or healing tape. Dressings for application in the treatment of fibrous hyperplasia, elevated, rounded, consistent and poorly defined borders occurring in the dermis and epidermis, usually after traumatic injury, burns, inflammatory and/or surgical processes, are widely known in the present status of the technique. Although these dressings have presented several drawbacks, such as: no reuse, low durability, absence of active ingredients that promote the perfect regeneration or restoration of the tissue, absence of compression in the scar, and high mobility of the skin in the scar place, that could promote its widening.

### SUMMARY OF THE INVENTION

The present invention has been developed in order to solve such problems, becoming indispensable for professionals and users, who use dressings or tapes to treat these hypertrophies and keloids, as well as in the enlargement prevention in the various phases of the cicatricial process. This invention is practical, safe and versatile, with a structure that enables a shorter healing time, allowing the absorption of active principles that favor healing and hydration, decrease pruritus and erythema, reduce the elevation of the scar due to the greater pressure in the central area. Also, it reduces the tension in the skin and consequently prevents the widening of the scar, aside from protecting the scar from invasion of bacteria. Other advantages of the compression are occlusion of small vessels within the scar, determining ischemia and reducing the number of fibroblasts, and the formation of collagen. In addition, the active principles allow the preservation of skin texture, hydration and emollience, with anti-oxidant, regenerating and healing action.

Thereby, the general conception of the present MANUFACTURING PROCESS FOR AN ADHERENT SILICONE HEALING TAPE/PAD/DRESSING OF SPECIFIC SHAPES, object of the present patent is quite effective and can be described as a more elevated or not, central inner core, of specific measures, with lowered external edges and knurled or with grooves or suction, or adhesive embossing, with or without micro-holes that surround the entire core to exert a gradual pressure on the skin. The invention is produced by mixing a compound A (Dimethylsiloxane, Dimethyl) and B (methylhydrogen siloxane) in equal proportions 1:1 and the tape being covered by a layer of glue consisting of a mixture A and B associated with several active ingredients, such as Vitamin E and Rosehip Oil, including biological dyes in the mixture to obtain various colors. It presents many action, such as immunostimulatory, moisturizing, anti-inflammatory, healing, regenerative, permeabilizing, anti-angiogenic, anti-wrinkle, antinociceptive, vasoconstricting, anti-pruritus, cosmetic, rejuvenating and preventive in terms of scar and stretch enlargement, and also has the function of stimulating and assisting reflex swallowing.

The tape/pad/dressing in contact with skin sends important information to the brain (sensory stimulation and proprioceptive information), reducing symptoms on muscle, facial and joint tissue, improving its functionality, reducing muscle fatigue and pain.

Different shapes, such as, curved, straight, rectangular and spherical, round, inverted T-shape, arc or U-shape, among other specific formats for each application, with micro holes or not, may be presented in different sizes (2×2 cm, 4×4 cm, 5×5 cm, 2×1 cm, 3×1 cm, 5×2.5 cm, 30×4 cm), among others, characterized by being soft, occlusive or semi-occlusive, with prolonged adherence, reusable and moldable to different regions of the body.

The invention is made of silicone with dye and glue with adhesive action, and has its own specific structure in the form of a single block in overlapping layers, with or without micro-holes. In order to provide an extremely practical, safe and accurate way for a complete optimization in the administration of hypertrophic scar and keloid prevention, as well as in reddish-new scar and burn scar, in the prevention of enlarged scar and stretch marks. The adhesive healing tape/pad/dressing can be used in various therapies (anti-inflammatory, anti-microbial, anti-wrinkle, anti-allergic, anti-erythema, antinoniceptiva, vasoconstrictor, etc), restoring or regenerating tissues, allied to a very low factor of contraindications and side effects. Because the tape/pad/dressing is anatomical, skin-resilient, highly resistant, with a specific moldable format, reusable and with greater practicality in handling and functionality.

### BRIEF DESCRIPTION OF THE DRAWINGS

The invention may be better understood from the following detailed description, in accordance with the attached figures, of which:

FIG. 1 represents a bottom view of the curved tape/pad/dressing; 1—Area with silicone and glue;

2—Area with silicone glue and compression;

3—Knurled, grooves or suctions and/or adhesive embossing;

FIG. 2 represents a top view of the curved tape/pad/dressing;

FIG. 3 represents a side view of the curved tape/pad/dressing;

FIG. 4 represents a bottom view of the flat horizontal tape/pad/dressing; 1—Area with glue;

2—Area with silicone glue and compression; 3—Knurled, grooves or suctions

FIG. 5 represents a top view of the flat horizontal tape/pad/dressing;

FIG. 6 represents a side view of the flat horizontal tape/pad/dressing;

FIG. 7 represents a bottom view of the rectangular tape/pad/dressing; 1—Area with glue; 2—Area with glue and compression; 3—Knurled, grooves or suctions;

FIG. 8 represents a top view of the rectangular tape/pad/dressing;

FIG. 9 represents a side view of the rectangular tape/pad/dressing;

FIG. 10 represents a bottom view of the spherical tape/pad/dressing; 1—Area with silicone and glue; 2—Area with silicone glue and compression; 3—Knurled, grooves or suctions; 4—Central area with a gap;

FIG. 11 represents a top view of the spherical tape/pad/dressing;

FIG. 12 represents a side view of the spherical tape/pad/dressing;

FIG. 13 represents a bottom view of the tape/pad/dressing in the inverted T-shape; 1—Area with silicone and glue; 2—Area with silicone glue and compression; 3—Knurled, grooves or suctions;

FIG. 14 represents a top view of the tape/pad/dressing in the inverted T-shape;

FIG. 15 represents a side view of the tape/pad/dressing in the inverted T-shape;

FIG. 16 represents a bottom view of the curved tape/pad/dressing for ear application; 1—Area with silicone and glue; 2—Area with silicone glue and compression; 3—Knurled, grooves or suctions;

FIG. 17 represents a top view of the curved tape/pad/dressing for ear application;

### DETAILED DESCRIPTION OF THE SEVERAL EMBODIMENTS

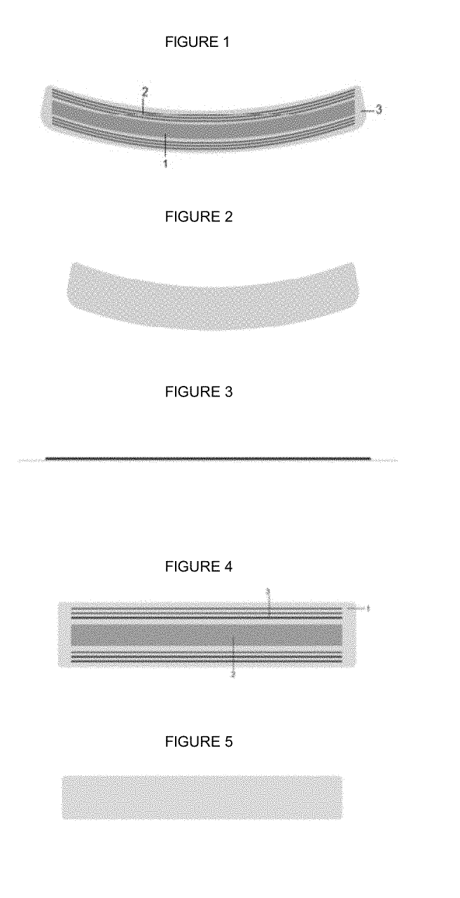

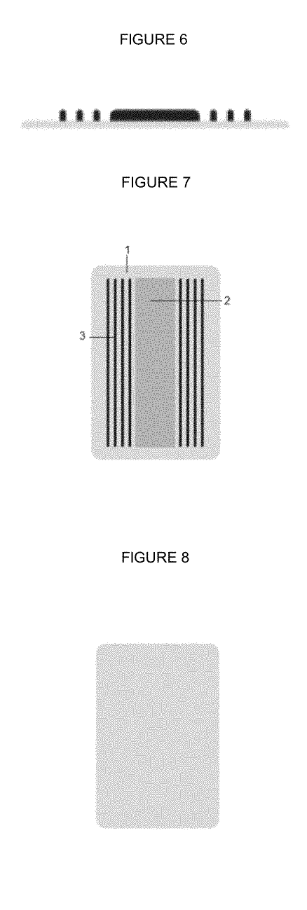

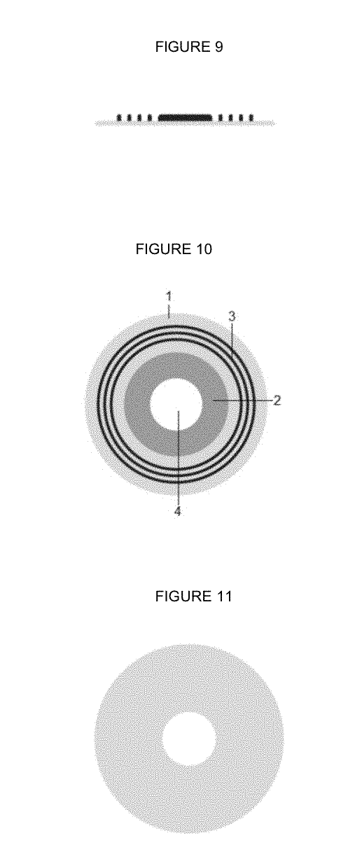

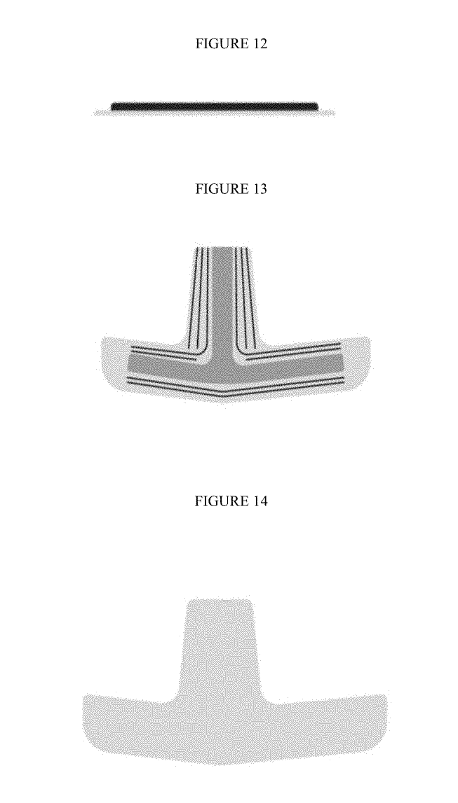

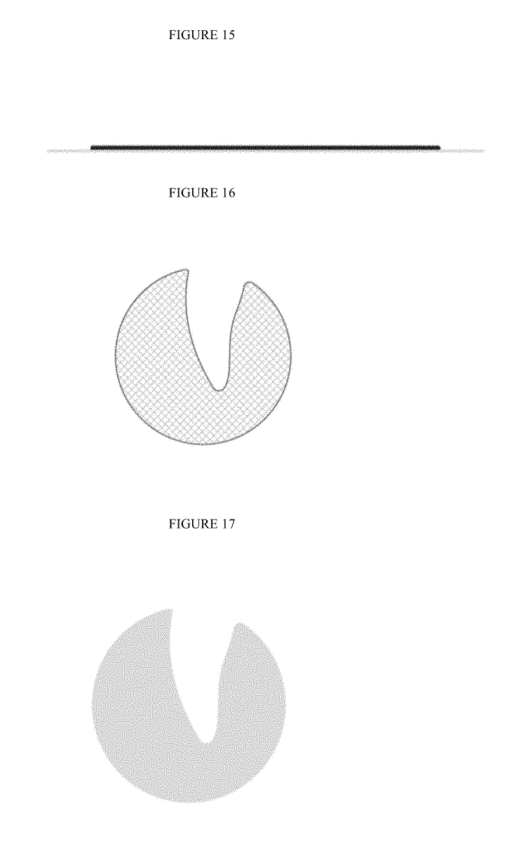

With reference to these figures, can be observed in the MANUFACTURING PROCESS FOR AN ADHERENT SILICONE HEALING TAPE/PAD/DRESSING OF SPECIFIC SHAPES, the silicone base area with glue (FIGS. 1 to 17), with a more elevated or not central area, with glue and compression (FIGS. 1 to 17 No. 2) with or without micro-holes which adheres to the skin through the knurl, grooves (FIG. 1 to 17 No. 3) or suction and adhesive embossing (FIGS. 1 to 17 No. 3);

The preparation protocol of the tape/pad/dressing consists of a single block in overlapping layers (FIG. 7 No. 1, 2, 3), following the example below:

Preparation of Silicone Tape

5.5 mL Silicone A

5.5 mL Silicone B

25 mg of dye

Mix well and place in the mold, incubating it at 65° C. to 80° C. for up to 5 min (3 min)

Preparation of the Glue

Mix in equal or unbalanced proportions to the volumes of:

40% to 60% Glue A and mix with the active ingredients (AI) A1 and A2 in the volume of 0.25 to 1% by PA.

40% to 60% Glue B

Mix well and incubate for 5 to 30 minutes until the bubbles are released from the ice. Spread on the tape homogeneously and incubate at 140° C. to 220° C. for up to 15 minutes, and then at 4° C. for up to 10 minutes. Leave at room temperature for another 15 to 20 min.

MANUFACTURING PROCESS FOR AN ADHERENT SILICONE HEALING TAPE/PAD/DRESSING OF SPECIFIC SHAPES according to the application needs and, as shown in the attached figures, can be comprised as an anatomical tape/pad/dressing (FIGS. 1,2,310,11,12, 13,14,15,16 and 17) which incorporates a structure containing integrated and symmetrically disposed in a single block in overlapping layers (FIG. 7 No. 1, 2, 3).

The tape/pad/dressing (FIGS. 1 to 17) should be applied with properly sanitized hands, the area to be treated must be clean and dry before applying it. If the area to be applied is oily, the oil should be removed so that the area is completely degreased, and the protective plastic must be removed and placed by slightly bending the body or approaching the skin in order to decrease the tension of it.

The adhesive tape/pad/dressing created for the prevention and treatment of recent and old stretch marks, wrinkle, enlarged scars, hypertrophic and keloid, is a self-adhesive mechanism that allows excellent support, reuse and adaptation to the affected area. The daily use tape can be washed with warm water and neutral soap, and must be dried before reuse. The tape should be used for approximately 30 to 60 days, which makes the product cost-effective.

The alleged Tape/Pad/Dressing (FIGS. 1 to 18) can be described as a more elevated or not central inner core (FIGS. 1 to 17, No.2), of specific measures; or with lowered external edges (FIGS. 1 to 17, No.1) that surround the entire core to exert pressure on the skin; or otherwise totally flat, produced by mixing a compound A (Dimethylsiloxane, Dimethyl) and B (methylhydrogen siloxane) in equal proportions 1:1, including dyes, chemical agents in the mixture to obtain various colors and therapeutic properties. The tape/pad/dressing is covered by a layer of glue consisting of an A and B mixture associated with active principles with the purpose of promoting healing, hydration, decrease of pruritus, decrease of erythema, reduction of scar elevation due to the increased pressure in the central area (FIGS. 1 to 17, No. 2), reducing the tension in the skin and consequently preventing the enlargement of the scar, protecting the scar from the invasion of bacteria. Other advantages of the compression are occlusion of small vessels within the scar, determining ischemia and reducing the number of fibroblasts and the formation of collagen. In addition, the active principles allow the preservation of skin texture, hydration and emollience, with anti-oxidant, regenerating and healing action.

Other actions are immunostimulatory, moisturizing, anti-inflammatory, healing, regenerative, permeabilizing, angiogenic, antinociceptive, vasoconstricting, anti-pruritus, cosmetic, rejuvenating and preventive in terms of scar enlargement, in various shapes (curved, flat, rectangular and spherical) (FIGS. 1 to 17) and sizes, specific for each application.

1. MANUFACTURING PROCESS FOR AN ADHERENT SILICONE HEALING TAPE/PAD/DRESSING OF SPECIFIC SHAPES characterized by a central inner core more elevated or not, with specific measures (FIGS. 1 to 17), with or without lowered external edges (FIG. 1 No. 3), which surround the entire core to exert a pressure (FIGS. 1 to 2) on the skin, produced by mixing a compound A (Dimethylsiloxane, Dimethyl and similar others) and B (methylhydrogen siloxane and similar others).

2. MANUFACTURING PROCESS FOR AN ADHERENT SILICONE HEALING TAPE/PAD/DRESSING OF SPECIFIC SHAPES accordingly to claim 1, characterize by a flat and single-layer mold (FIGS. 1 to 17), with or without knurls or grooves or suction (FIGS. 1 to 17 No.2), without lowered external edges. The invention is produced mixing a compound A (Dimethylsiloxane, Dimethyl and similar others) and B (methylhydrogen siloxane and similar others) in equal proportions of 1:1. Biological dyes are included in the mixture to obtain various colors. The invention is placed in shape and incubated at 65° C. to 80° C. for up to 5 minutes and covered by a layer of glue with medium adhesion in the variable proportion of 40% to 60% glue from the mixture of components A and B of the glue with active ingredients (vitamin E, rosehip oil, among others) in the proportion of 0.1% to 3% of the volume of glue, spread on the tape (FIGS. 1 to 17) homogeneously (still warm) and incubated at 140° C. to 220° C. for 15 min. Then, it is cooled to 4° C. for 5 min, in order to exert slight pressure on the skin for purposes of wound management, post-surgical treatments, cosmetic and skin diseases.

3. MANUFACTURING PROCESS FOR AN ADHERENT SILICONE HEALING TAPE/PAD/DRESSING OF SPECIFIC SHAPES accordingly to claim 1, characterized by a tape/dressing without inner core, or flat, with a layer of glue with medium adhesion (FIG. 1 a 17).

4. MANUFACTURING PROCESS FOR AN ADHERENT SILICONE HEALING TAPE/PAD/DRESSING OF SPECIFIC SHAPES accordingly to claim 1, characterize by a layer of glue with medium adhesion (FIG. 1 to 17 No.1, 2, 3) containing synthetic chemical and biological agents, and several actions, such as: immunostimulatory, moisturizing, anti-inflammatory, healing, regenerative, permeabilizing, anti-angiogenic,
antinociceptive, anti-pruritus, cosmetic and rejuvenating, vasoconstricting, and preventive activity of scar enlargement.

5. MANUFACTURING PROCESS FOR AN ADHERENT SILICONE HEALING TAPE/PAD/DRESSING OF SPECIFIC SHAPES characterized by a central inner core more elevated or not, with specific measures (FIGS. 1 to 17), with or without lowered external edges (FIG. 1 No. 3), which surround the entire core to exert a pressure (FIGS. 1 to 2) on the skin, produced by mixing a compound A (Dimethylsiloxane, Dimethyl and similar others) and B (methylhydrogen siloxane and similar others).

6. MANUFACTURING PROCESS FOR AN ADHERENT SILICONE HEALING TAPE/PAD/DRESSING OF SPECIFIC SHAPES accordingly to claim 1, characterize by a flat and single-layer mold (FIGS. 1 to 17), with or without knurls or grooves or suction (FIGS. 1 to 17 No.2), without lowered external edges. The invention is produced mixing a compound A (Dimethylsiloxane, Dimethyl and similar others) and B (methylhydrogen siloxane and similar others) in equal proportions of 1:1. Biological dyes are included in the mixture to obtain various colors. The invention is placed in shape and incubated at 65° C. to 80° C. for up to 5 minutes and covered by a layer of glue with medium adhesion in the variable proportion of 40% to 60% glue from the mixture of components A and B of the glue with active ingredients (vitamin E, rosehip oil, among others) in the proportion of 0.1% to 3% of the volume of glue, spread on the tape (FIGS. 1 to 17) homogeneously (still warm) and incubated at 140° C. to 220° C. for 15 min. Then, it is cooled to 4° C. for 5 min, in order to exert slight pressure on the skin for purposes of wound management, post-surgical treatments, cosmetic and skin diseases.

7. MANUFACTURING PROCESS FOR AN ADHERENT SILICONE HEALING TAPE/PAD/DRESSING OF SPECIFIC SHAPES accordingly to claim 1, characterized by a tape/dressing without inner core, or flat, with a layer of glue with medium adhesion (FIG. 1 a 17).

8. MANUFACTURING PROCESS FOR AN ADHERENT SILICONE HEALING TAPE/PAD/DRESSING OF SPECIFIC SHAPES accordingly to claim 1, characterize by a layer of glue with medium adhesion (FIG. 1 to 17 No.1, 2, 3) containing synthetic chemical and biological agents, and several actions, such as: immunostimulatory, moisturizing, anti-inflammatory, healing, regenerative, permeabilizing, anti-angiogenic, antinociceptive, anti-pruritus, cosmetic and rejuvenating, vasoconstricting, and preventive activity of scar enlargement.

9. MANUFACTURING PROCESS FOR AN ADHERENT SILICONE HEALING TAPE/PAD/DRESSING OF SPECIFIC SHAPES accordingly to claim 1, characterize by the cure of the adhesive heating mixture, with or without ventilation, with or without vacuum, carried out in a common oven, microwave, in the presence or absence of ultraviolet light and/or in infrared light.

10. MANUFACTURING PROCESS FOR AN ADHERENT SILICONE HEALING TAPE/PAD/DRESSING OF SPECIFIC SHAPES accordingly to claim 1, characterize by being manufactured in several formats: (FIG. 1 to 17) curved, straight, rectangular, round, U-shaped, inverted T-shaped, arch and concave, with open/closed cores (FIG. 10, 11,12), with or without internal elevation, knurled, with grooves, suction or flat (FIG. 1 to 17 No.3).
